(19) United States
(12) Patent Application Publication
(10) Pub. No.: US 2023/0030941 A1
(43) Pub. Date: Feb. 2, 2023

(54) ULTRASOUND IMAGING SYSTEM AND METHOD FOR USE WITH AN ADJUSTABLE NEEDLE GUIDE

(71) Applicant: GE Precision Healthcare LLC, Wauwatosa, WI (US)

(72) Inventor: Bong Hyo Han, Seongnam-si (KR)

(21) Appl. No.: 17/388,192

(22) Filed: Jul. 29, 2021

(51) Int. Cl. A61B 17/34; A61B 8/00

(52) U.S. Cl. CPC A61B 17/3403; A61B 8/4455; A61B 8/465; A61B 8/467; A61B 8/463; A61B 2017/3405; A61B 2017/3413

(57) ABSTRACT

Various methods and ultrasound imaging systems are provided for automatically calculating and displaying a setting for an adjustable needle guide. An exemplary method includes acquiring an image including a target with an ultrasound probe and displaying the image on a display device. The method includes receiving an input, via a user interface, identifying the target in the image. The method includes automatically calculating, with a processor, the setting for an adjustable needle guide that is configured to be attached to the ultrasound probe based on the identified target in the image, wherein the setting is at least one of an angle measurement for the adjustable needle guide or one of a plurality of angular positions for the adjustable needle guide. The method includes displaying the setting for the adjustable needle guide on the display device.

### FIELD OF THE INVENTION

This disclosure relates generally to an ultrasound imaging system and method of using the ultrasound imaging system with an adjustable needle guide.

### BACKGROUND OF THE INVENTION

Some medical procedures, such as obtaining a biopsy or applying local anesthesia require the accurate positioning of a needle within a target structure. For example, it may be desired to obtain a tissue biopsy from a region of a suspected tumor or lesion. Or, when administering a nerve block to a patient, it is important to position the needle within the desired nerve before administering anesthesia via the needle. In addition to inserting the needle in the desired anatomical structure, it is extremely important to avoid accidentally damaging other critical organs or structures during the process of inserting the needle and/or positioning the needle in the desired anatomical structure. For these and other reasons, it is common to use ultrasound to assist with needle placement for a number of procedures.

According to conventional techniques for ultrasound-guided needle placement, it is known to use a needle guide attached to the ultrasound probe. Using a needle guide has been shown to increase the speed and accuracy of positioning a needle in the desired anatomical structure or tissue. Additionally, when acquiring two-dimensional ultrasound images, a needle guide may help to keep the needle visible within the imaging plane while the needle is being inserted.

Conventional needle guides may be either at a fixed angle with respect to the ultrasound probe, or needle guides may be adjustable so that they may be positioned at two or more different angles with respect to the ultrasound probe. However, according to conventional techniques, the needle guide is set to a fixed angle with respect to the ultrasound probe and then the clinician attempts to position the ultrasound probe into position where the anticipated needle path will intersect the structure of interest. One significant issue with the conventional technique is that the clinician may be required to tip the ultrasound probe at an angle with respect to the skin surface of the patient in order to align the anticipated needle path with the structure of interest. This may result in an ultrasound probe position that is less stable and therefore more difficult for the clinician to hold the ultrasound probe in a fixed position while inserting the needle. Additionally, tilting the ultrasound probe may result in poor probe contact with the patient which, in turn, may result in poor image quality.

Conventional techniques using an adjustable needle guide require the clinician to make a best estimate of the appropriate angle for the adjustable needle guide. As such, it may still take the clinician an unduly long time in order to locate the desired anatomical structure with the needle.

Therefore, for these and other reasons, an improved method and ultrasound imaging system for use with an adjustable needle guide is desired.

### BRIEF DESCRIPTION OF THE INVENTION

The above-mentioned shortcomings, disadvantages and problems are addressed herein which will be understood by reading and understanding the following specification.

In an embodiment, a method for determining a setting of an adjustable needle guide includes acquiring an image including a target with an ultrasound probe. The method includes displaying the image on a display device and receiving an input, via a user interface, identifying the target in the image. The method includes automatically calculating, with a processor, the setting for the adjustable needle guide based on the identified target in the image, wherein the setting is at least one of an angle measurement for the adjustable needle guide or one of a plurality of angular positions for the adjustable needle guide, wherein the setting is configured to position the adjustable needle guide to guide a needle to the target. The method includes displaying the setting on the display device.

In another embodiment, an ultrasound imaging system includes an ultrasound probe, a display device, a user interface, and a processor. The processor is configured to control the ultrasound probe to acquire an image including a target, display the image on the display device, receive an input, via the user interface, identifying the target in the image, and automatically calculate a setting for an adjustable needle guide that is configured to be attached to the ultrasound probe based on the identified target in the image, wherein the setting is at least one of an angle measurement for the adjustable needle guide or an indication of one of a plurality of angular positions for the adjustable needle guide. The processor is configured to display the setting on the display device.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is an exploded representation of touchscreen in accordance with an embodiment;

FIG. 3 is a flow chart of a method in accordance with an embodiment;

FIG. 4 is a screenshot in accordance with an embodiment;

FIG. 5 is a screenshot in accordance with an embodiment;

FIG. 6 is representation of an ultrasound probe with an adjustable needle guide in accordance with an embodiment;

FIG. 7 is representation of an ultrasound probe with an adjustable needle guide in accordance with an embodiment;

FIG. 8 is representation of an ultrasound probe with an adjustable needle guide in accordance with an embodiment;

FIG. 9 is a representation of an ultrasound probe, an adjustable needle guide, and an image in accordance with an embodiment; and

FIG. 10 is a triangle in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized, and that logical, mechanical, electrical and other changes may be made without departing from the scope of the embodiments. The following detailed description is, therefore, not to be taken as limiting the scope of the invention.

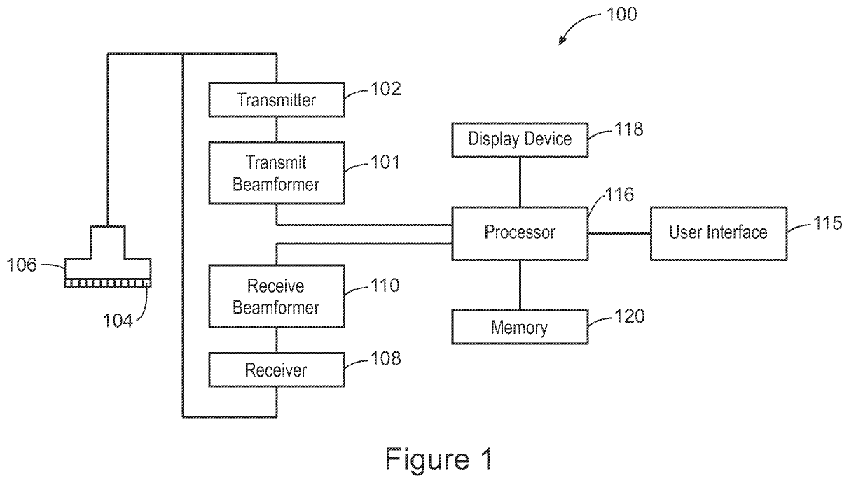

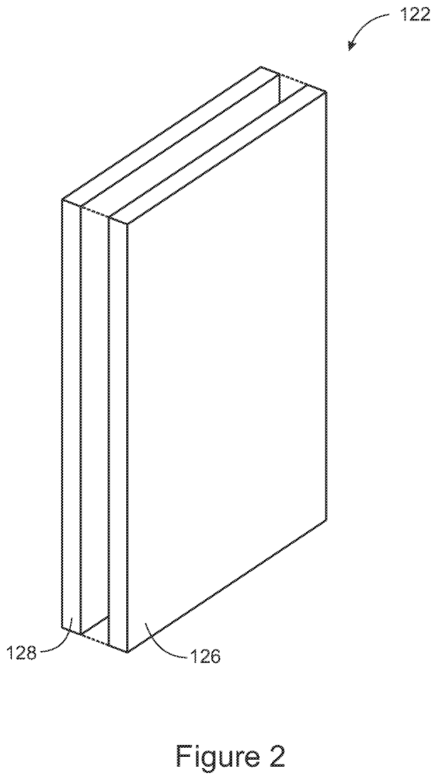

FIG. 1 is a schematic diagram of an ultrasound imaging system 100 in accordance with an embodiment. The ultrasound imaging system 100 includes a transmit beamformer 101 and a transmitter 102 that drive elements 104 within an ultrasound probe 106 to emit pulsed ultrasonic signals into a body (not shown) through one or more transmit events. The ultrasound probe 106 may be any type of ultrasound probe. For example, the ultrasound probe 106 may be a linear array probe, a curved-linear array probe, a convex array probe, a phased array probe, a 2D matrix array probe capable of 3D or 4D scanning, a mechanical 3D probe, etc. Still referring to FIG. 1, the pulsed ultrasonic signals are back-scattered from structures in the body to produce echoes that return to the elements 104. The ultrasound probe 106 may be in electrical communication with one or more other components of the ultrasound imaging system 100 via wired and/or wireless techniques. The echoes are converted into electrical signals by the elements 104 and the electrical signals are received by a receiver 108. The electrical signals representing the received echoes are passed through a receive beamformer 110 that outputs ultrasound data. According to some embodiments, the ultrasound probe 106 may contain electronic circuitry to do all or part of the transmit beamforming and/or the receive beamforming. For example, all or part of the transmit beamformer 101, the transmitter 102, the receiver 108 and the receive beamformer 110 may be situated within the ultrasound probe 106. According to some embodiments, the ultrasound probe 106 may be configured to wirelessly communicate with a phone-sized or tablet-sized device and the ultrasound probe and either the phone-sized device or the tablet-sized device may collectively perform all the functions associated with the elements identified on FIG. 1. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system 100. A user interface 115 may be used to control operation of the ultrasound imaging system 100. The user interface 115 may be used to control the input of patient data, or to select various modes, operations, parameters, and the like. The user interface 115 may include one or more user input devices such as a keyboard, hard keys, a touch pad, a track ball, rotary controls, sliders, soft keys, or any other user input devices. According to some embodiments, the user interface 115 may include a touch panel that is part of a touchscreen. An exemplary touchscreen will be described hereinafter with respect to FIG. 2.

The ultrasound imaging system 100 includes a display device 118. The display device 118 may include any type of display screen or display that is configured to display images, text, graphical user interface elements, etc. The display device 118 may be, for example, a cathode ray tube (CRT) display, a light-emitting diode (LED) display, an organic light-emitting diode (OLED) display, a liquid crystal display (LCD), etc. According to some embodiments, the display device 118 may be a display screen that is a component of a touchscreen.

As discussed above, the display device 118 and the user interface 115 may be components in a touchscreen. FIG. 2 is an exploded representation of a touchscreen 122 in accordance with an exemplary embodiment. The touchscreen 122 includes a touch panel 126 and a display screen 128 in accordance with an embodiment. The touch panel 126 may be located behind the display screen 128 or in front of the display screen 128 according to various non-limiting examples. For embodiments where the touch panel 126 is positioned in front of the display screen 128, the touch panel 126 may be configured to be substantially transparent so that the user may see images displayed on the display screen 128. The touch panel 126 may utilize any type of technology configured to detect a touch or gesture applied to the touch panel 126 of the touchscreen 122. As discussed hereinabove, the display device 118 may include a display screen of a touchscreen such as the display screen 128, and the user interface 115 may include a touch panel, such as the touch panel 126 of the touchscreen 122. The touch panel 126 may be configured to detect single-point touch inputs and/or multi-point touch inputs according to various embodiments. The touch panel 126 may utilize any type of technology configured to detect a touch or gesture applied to the touch panel 126 of the touchscreen 122. For instance, the touch panel 126 may include resistive sensors, capacitive sensors, infrared sensors, surface acoustic wave sensors, electromagnetic sensors, near-filed imaging sensor, or the like. Some embodiments may utilize the touch panel 126 of the touchscreen 122 to provide all of the user interface functionalities for the ultrasound imaging system 100, while other embodiments may also utilize one or more other components as part of the user interface 115.

Referring back to FIG. 1, the ultrasound imaging system 100 also includes a processor 116 to control the transmit beamformer 101, the transmitter 102, the receiver 108 and the receive beamformer 110. The user interface 115 is in electronic communication with the processor 116. The processor 116 may include one or more central processing units (CPUs), one or more microprocessors, one or more microcontrollers, one or more graphics processing units (GPUs), one or more digital signal processors (DSP), and the like. According to some embodiments, the processor 116 may include one or more GPUs, where some or all of the one or more GPUs include a tensor processing unit (TPU). According to embodiments, the processor 116 may include a field-programmable gate array (FPGA), or any other type of hardware capable of carrying out processing functions. The processor 116 may be an integrated component or it may be distributed across various locations. For example, according to an embodiment, processing functions associated with the processor 116 may be split between two or more processors based on the type of operation. For example, embodiments may include a first processor configured to perform a first set of operations and a second, separate processor to perform a second set of operations. According to embodiments, one of the first processor and the second processor may be configured to implement a neural network. The processor 116 may be configured to execute instructions accessed from a memory. According to an embodiment, the processor 116 is in electronic communication with the ultrasound probe 106, the receiver 108, the receive beamformer 110, the transmit beamformer 101, and the transmitter 102. For purposes of this disclosure, the term “electronic communication” may be defined to include both wired and wireless connections. The processor 116 may control the ultrasound probe 106 to acquire ultrasound data. The processor 116 controls which of the elements 104 are active and the shape of a beam emitted from the ultrasound probe 106. The processor 116 is also in electronic communication with a display device 118, and the processor 116 may process the ultrasound data into images for display on the display device 118. According to embodiments, the processor 116 may also include a complex demodulator (not shown) that demodulates the RF data and generates raw data. In another embodiment the demodulation can be carried out earlier in the processing chain. The processor 116 may be adapted to perform one or more processing operations according to a plurality of selectable ultrasound modalities on the data. The data may be processed in real-time during a scanning session as the echo signals are received. The processor 116 may be configured to scan-convert the ultrasound data acquired with the ultrasound probe 106 so it may be displayed on the display device 118. Displaying ultrasound data in real-time may involve displaying the ultrasound data without any intentional delay. For example, the processor 116 may display each updated image frame as soon as each updated image frame of ultrasound data has been acquired and processed for display during the display of a real-time image. Real-time frame rates may vary based on the size of the region or volume from which data is acquired and the specific parameters used during the acquisition. According to other embodiments, the data may be stored temporarily in a buffer (not shown) during a scanning session and processed in less than real-time. According to embodiments that include a software beamformer, the functions associated with the transmit beamformer 101 and/or the receive beamformer 110 may be performed by the processor 116.

According to an embodiment, the ultrasound imaging system 100 may continuously acquire ultrasound data at a frame-rate of, for example, 10 Hz to 30 Hz. Images generated from the data may be refreshed at a similar frame-rate. Other embodiments may acquire and display data at different rates. For example, some embodiments may acquire ultrasound data at a frame rate of less than 10 Hz or greater than 30 Hz depending the size of each frame of data and the parameters associated with the specific application. For example, many applications involve acquiring ultrasound data at a frame rate of about 50 Hz. A memory 120 is included for storing processed frames of acquired data. In an exemplary embodiment, the memory 120 is of sufficient capacity to store frames of ultrasound data acquired over a period of time at least several seconds in length. The frames of data are stored in a manner to facilitate retrieval thereof according to its order or time of acquisition. The memory 120 may comprise any known data storage medium.

In various embodiments of the present invention, data may be processed by other or different mode-related modules by the processor 116 (e.g., B-mode, color Doppler, M-mode, color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate and combinations thereof, and the like. The image beams and/or frames are stored, and timing information indicating a time at which the data was acquired in memory may be recorded. The modules may include, for example, a scan conversion module to perform scan conversion operations to convert the image frames from beam space coordinates to display space coordinates. A video processor module may be provided that reads the image frames from a memory, such as the memory 120, and displays the image frames in real time while a procedure is being carried out on a patient. The video processor module may store the image frames in an image memory, from which the images are read and displayed.

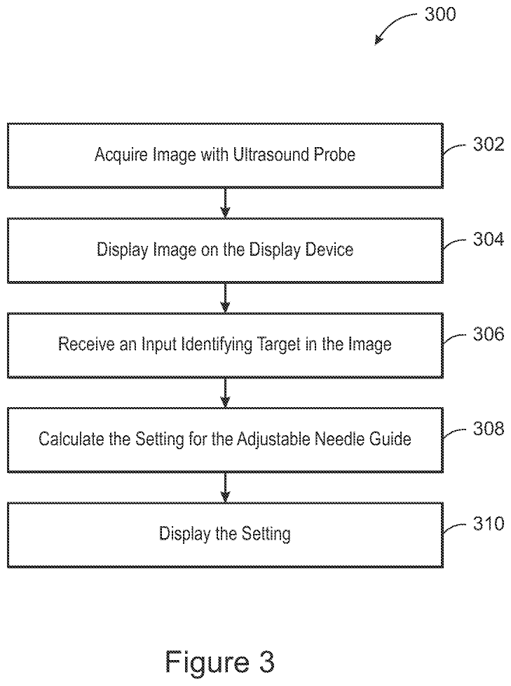

FIG. 3 illustrates a flowchart of an embodiment of a method 300 for determining and displaying a setting for an adjustable needle guide. The method 300 shown in FIG. 2 may be performed with the ultrasound imaging system 100 shown in FIG. 1 according to an exemplary embodiment. The technical effect of the method 300 is the calculation and display of a setting for the adjustable needle guide based on the identification of the target in the image.

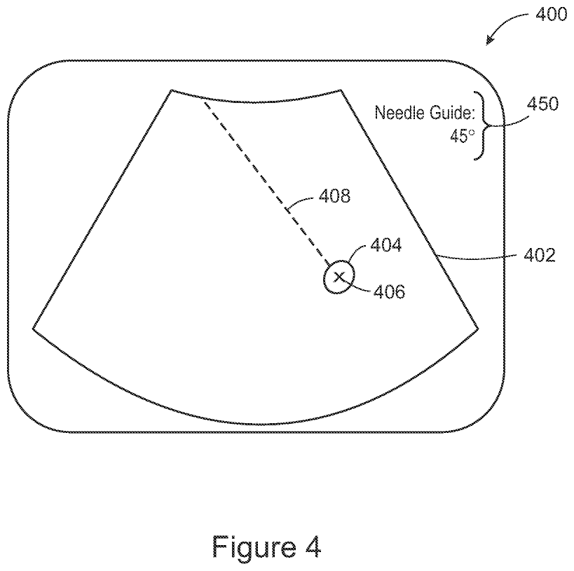

FIG. 4 is a representation of a screenshot 400 in accordance with an embodiment. The screenshot 400 may be displayed on the display device 118. The screenshot 400 includes an image 402.

At step 302, the processor 116 controls the ultrasound probe 106 to acquire an image of a portion of a patient. According to an exemplary embodiment, the ultrasound probe 106 may be used to acquire the image 402 depicted in FIG. 4. The image 402 may be acquired using any ultrasound imaging mode, but according to an exemplary embodiment, the image 402 may be a B-mode image.

At step 304, the processor 116 displays the image 300 on the display device 118.

At step 306, the processor 116 receives an input from the user interface 115 identifying a target in the image. The target is the anatomical location where the clinician would like to position a tip of a needle. While not shown on the flow chart 300, according to various embodiments, the clinician may need to first enter a needle-specific mode before the processor 116 is configured to receive the input from the touchscreen. For example, in the embodiment represented by FIG. 4, the image 402 includes a target 404. The target 404 represents a desired anatomical target or location for a needle that is inserted into the patient's tissue. It may be desired to guide the needle to the target 404 for a variety of reasons, such as to obtain a biopsy of tissue from the target 404, to inject a substance into the target 404, or to piece or puncture the target 404. For example, it may be desirable to obtain a biopsy from a polyp, a tumor, a suspected polyp, or a suspected tumor in order to determine more detailed information about the type of tissue. According to these embodiments, the target 404 may be a polyp, a tumor, a suspected polyp, a suspected tumor, or any other structure from which a biopsy is desired. The target 404 may be, for example, a nerve according to embodiments where a nerve block is performed. The needle may be used to administer anesthetic or a numbing agent to the nerve in order to decrease pain and/or as part of a surgical procedure. The target 304 may be any other structure to which it is desired to administer a targeted dose of medicine according to various embodiments.

At step 306, the processor 116 receives an input identifying a target, such as the target 404, in the image 402. According to an embodiment, the clinician may use the user interface 115 to position a visual indicator 406, such as a cursor or pointer, on the target 404. The visual indicator 406 is shown as an “X” in FIG. 3, but the visual indicator may be different according to other embodiments. For example, the visual indicator may be a crosshairs, a plus sign (“+”), a polygon, such as a triangle, a square, a rectangle, etc., a circle, an arrow, or any other type of marker according to various embodiments. According to some embodiments, the visual indicator may be configured to pulse or strobe, or to blink while it is displayed on the display device 118. According to some embodiments that include a touchscreen, the visual indicator 406 may be offset from the location of the touch input in order to allow the clinician to more clearly see the location of the visual indicator with respect to the image 402 while in the process of interfacing with the touchscreen 122.

As described hereinabove, in some embodiments, both the display device 118 and the user interface 115 may be combined in a touchscreen, such as the touchscreen 122. According to an exemplary embodiment where the ultrasound imaging system includes the touchscreen 122, receiving an input identifying a target, such as the target 404, may include receiving a touch input through the touchscreen 122. For example, the user may simply touch an area of the touchscreen 122 at the location of the designated target. According to some embodiments, the processor 116 may be configured to display a visual indicator, such as the visual indicator 406, at the location indicated by the touch input. According to various embodiments, the processor 116 may be configured to allow the clinician to reposition the visual indicator 406 to a different location by dragging the visual indicator 406 to a different location on the image 402. The processor 116 may use the last location of the visual indicator 406 to identify target or the processor 116 may be configured to designate the location of the target in response to an additional user input, such as a button press or a touch gesture, such as, for example a tap gesture or a double-tap gesture.

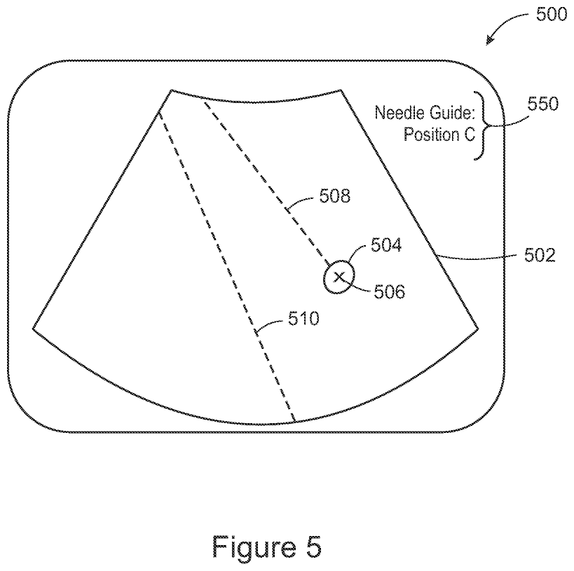

FIG. 5 is a representation of a screenshot 500 in accordance with an exemplary embodiment. The screenshot 500 includes an image 502, a target 504, a visual indicator 506, an expected guideline 508 and a base guideline 510. The image 502, the target 504, the visual indicator 506, and the expected guideline 508 shown in FIG. 5 correspond to the image 402, the target 404, the visual indicator 406, and the expected guide line 508 respectively that were shown in FIG. 4 and therefore will not be described in additional detail. The screenshot 500 also includes a base guide line 510. The base guide line 510 may be at a fixed position or a known position with respect to the ultrasound probe 106. The guide lines (508, 510) will be described in more detail hereinafter.

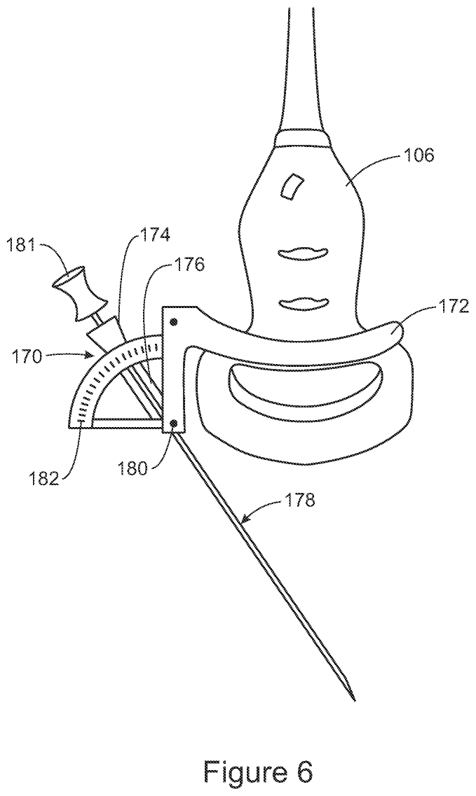

FIG. 6 is a representation of the ultrasound probe 106 and adjustable needle guide 170 in accordance with an exemplary embodiment. The adjustable needle guide 170 is shown in a configuration where is it attached to the ultrasound probe 106 via a bracket 172. The adjustable needle guide 170 includes a needle cone 174 and a guide portion 176 that is configured to receive a needle, such as the needle 178, which includes a grip 181. The adjustable needle guide 170 is configured to be positioned in a plurality of different positions by pivoting about pivot 180. The adjustable needle guide 170 includes an angle guide 182. According to an embodiment, such as that shown in FIG. 6, the angle guide 182 may include a plurality of different marks, where each mark represent a different angular position. Some or all of the marks may be labeled with an angular measurement (45 degrees, 50 degrees, 55 degrees, etc.) according to various embodiments. The adjustable needle guide 170 is configured to be adjustable in order to position the needle 178 at different positions with respect to the ultrasound probe 106.

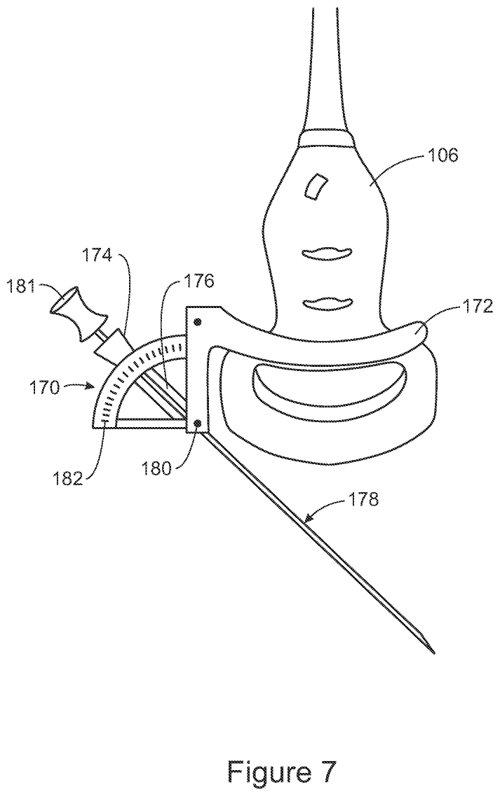

FIG. 7 is a representation of the ultrasound probe 106 and the adjustable needle guide 170, with the adjustable needle guide 170 in a different setting than that shown in FIG. 6 in accordance with an exemplary embodiment. FIG. 6 shows the adjustable needle guide 170 adjusted to a first angular position and FIG. 7 shows the adjustable needle guide 170 adjusted to a second angular position.

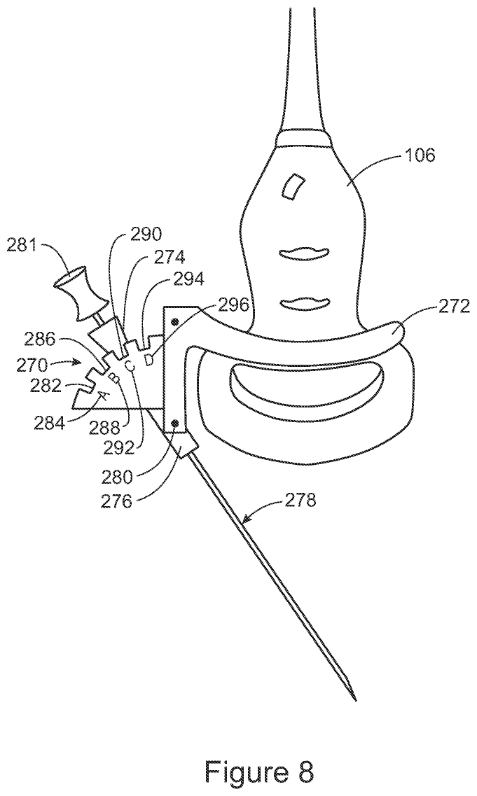

FIG. 8 is a representation of the ultrasound probe 106 and an adjustable needle guide 270 according to an exemplary embodiment. The adjustable needle guide 270 is different from the adjustable needle guide 170 shown in FIGS. 6 and 7. The adjustable needle guide 270 includes a bracket 272 for attachment to the ultrasound probe 106. The adjustable needle guide 270 includes a needle cone 274 for receiving a needle such as the needle 278. The adjustable needle guide 270 also includes a guide portion 276 for controlling the position of the needle 278 with respect to the adjustable needle guide 270, and hence, with respect to the ultrasound probe 106. The needle 278 includes a grip 281. The adjustable needle guide 270 may include a plurality of indexed angular positions. The clinician may adjust the adjustable needle guide 270 to any one of the indexed angular positions in order to adjust the angle of the guide portion 276 with respect to the ultrasound probe 106. For example, the adjustable needle guide 270 is shown with four indexed angular positions labeled “A,” “B,” “C,” and “D”. The adjustable needle guide 270, for instance, includes a first indexed angular position 282 labeled with a first marker 284, which is “A” in the embodiment shown in FIG. 8. The adjustable needle guide 270 includes a second indexed angular position 286 labeled with a second marker 288, which is a “B” in the embodiment shown in FIG. 8. The adjustable needle guide 270 includes a third indexed angular position 290 labeled with a third marker 292, which is a “C” in the embodiment shown in FIG. 8. The adjustable needle guide 270 includes a fourth indexed angular position 294 labeled with a fourth marker 296, which is a “D” in the embodiment shown in FIG. 8. In other embodiments, the adjustable needle guide may include a different number of indexed angular positions and/or each indexed angular position may be labeled differently. For example, according to an embodiment, each indexed angular position may be labeled with a number (such as “1,” “2,” “3”, etc.) instead of letters as shown in FIG. 8.

At step 308, the processor 116 calculates a setting for the adjustable needle guide (170, 270) based on the target identified in the image, such as the target 404 identified in the image 402 in FIG. 4. According to an exemplary embodiment, the adjustable needle guide (170, 270) may be attached to the ultrasound probe 106 during step 302 when the image (402, 502) is acquired. Identification information regarding the adjustable needle guide (170, 270) is provided to the processor 116. The information may be manually provided to the processor 116, such as via inputs through the user interface 115, or the information may be automatically provided to the processor 116 through wired or wireless techniques. For example, according to an embodiment, the adjustable needle guide (170, 270) may include a wireless identification chip, such as a radio-frequency identification (RFID) chip, that is detected by an RFID reader installed on the ultrasound imaging system 100 that is in electronic communication with the processor 116. According to other embodiments, the adjustable needle guide (170, 270) may include other types of identifiers, such as a bar code, a QR code, etc. and the ultrasound imaging system 100 may include a reader configured to scan the identifier (e.g., bar code, QR code, etc.) in order to receive identification for the adjustable needle guide (170, 270). The identification may include information such as the make and model of the adjustable needle guide (170, 270), or the identification information may just include geometrical information regarding the position of the adjustable needle guide (170, 270) with respect to the ultrasound probe 106 when the adjustable needle guide (170, 270) is attached to the ultrasound probe 106.

According to some embodiments, the manufacturer of the adjustable needle guide (170, 270) may provide a base guide line at a fixed position with respect to the adjustable needle guide (170, 270). The base guide line 510 shown in FIG. 5 is an example of a base guide line with respect to an adjustable needle guide (170, 270). The base guideline 510 is in a known, calibrated position with respect to the adjustable needle guide (170, 270). As such, after identification information for the adjustable needle guide (170, 270) has been provided to the processor 116, the processor 116 can display the base guide line 510 on an image, such as the image 502. According to various embodiments, the processor 116 may, for example, access a look-up table in order to determine the position of the base guide line with respect to a particular adjustable needle guide.

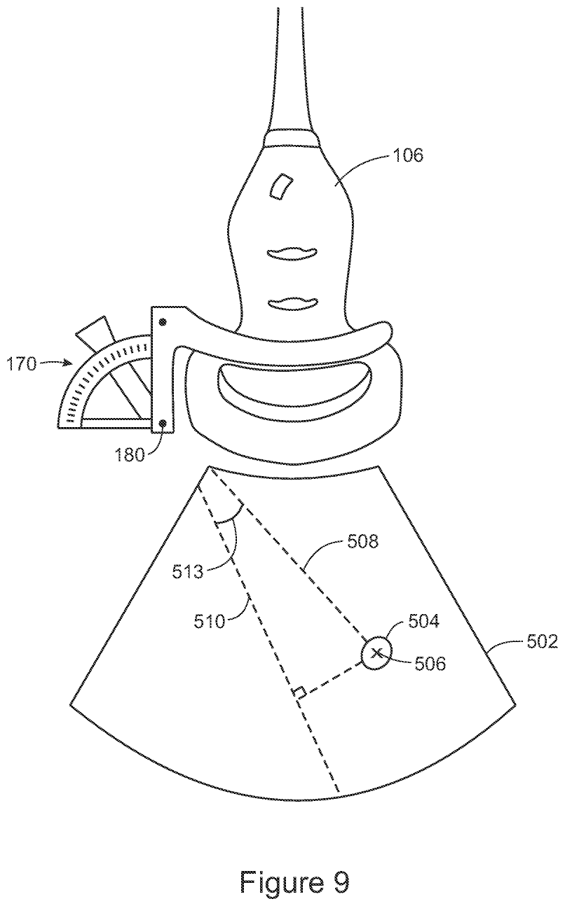

FIG. 9 is a schematic illustration of an ultrasound probe and an adjustable needle guide with respect to an image in accordance with an exemplary embodiment. FIG. 9 includes the ultrasound probe 106, and the adjustable needle guide 170 with respect to the image 502 and will be used to illustrate how the processor 116 may calculate a setting for an adjustable needle guide in accordance with an embodiment. FIG. 9 shows the ultrasound probe 106 and the adjustable needle guide 170 in relation to the image 502 at the time the image 502 is acquired. The image 502 includes the base guide line 510 and the expected guide line 508. As discussed hereinabove, the base guide line 510 is in a known, calibrated position with respect to the adjustable needle guide 170. It should be appreciated that FIG. 9 shows one example of a base guideline and that in other embodiments, the base guide line may be positioned at a different position with respect to a respective adjustable needle guide. In other embodiments, a graphical representation of the base guide line may not be displayed on the image. The image 502 also includes the expected guideline 508. As discussed previously, the expected guideline 508 is generated based on the user-selected position of the target 504 and represents the desired path for the needle once the adjustable needle guide has been adjusted according to the appropriate setting.

Once the adjustable needle guide, such as the adjustable needle guide 170, has been identified by the processor 116, the processor 116 may obtain information regarding the position of the adjustable needle guide 170 with respect to the ultrasound probe 106, which in turn may be used to calculate/determine the relative position of the adjustable needle guide 170 with respect to the image, such as the image 502. The processor 116 may use this information to calculate a setting for the adjustable needle guide 170. For example, since the relative position of the ultrasound probe 106 is known with respect to the adjustable needle guide 170, and the relative position of the image 502 is known with respect to the ultrasound probe 106, the processor 116 may be configured to calculate a geometric transformation in order to calculate a setting for the adjustable needle guide 170 in order to cause the needle to follow the expected guide line 508 represented in the image 502. According to an exemplary embodiment, the processor 116 may use a base guide line associated with the adjustable needle guide 170, such as the base guide line 510, in order to calculate the setting for the adjustable needle guide 170.

For example, both the base guide line 510 and the expected guide line 508 intersect at a predetermined point on the adjustable needle guide 170. According to the embodiment shown in FIG. 7, the base guide line 510 and the expected guide line 508 intersect at the pivot 180. According to other embodiments that base guide line and the expected guide line may intersect at a different predetermined point or location with respect to the adjustable needle guide. The processor 116 may use the location of the predetermined point, such as the pivot 180, on the adjustable needle guide 170 in order to determine how to display the expected guide line 508 on the image 502. In other words, the processor 116 may determine where to display the expected guide line 508 on the image 502 by positioning a line that would connect the target 504 to a predetermined location on the adjustable needle guide 170, such as the pivot 180.

The processor 116, may, for instance, calculate an angle 513 between the base guide line 510 and the expected guide line 508. The angle 513 represents the angular difference between the base guide line 510 and the expected guide line 508. The processor 116 may, for instance, calculate the angle 513 between the base guide line 510 and the expected guide line 508 using trigonometry.

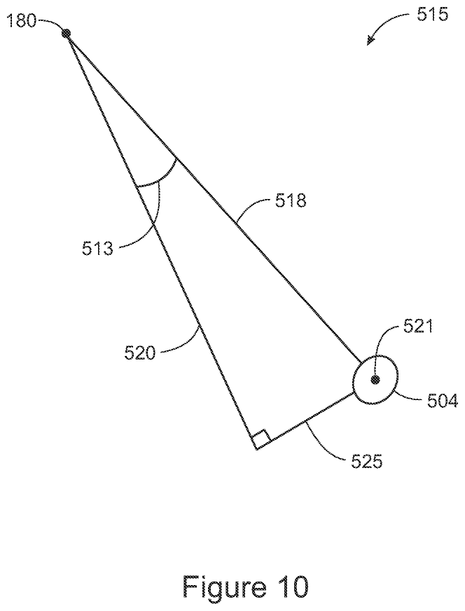

FIG. 10 is a representation of a triangle 515 that will be used to explain how trigonometry may be used to calculate the setting for an adjustable needle guide in accordance with an embodiment. The triangle 515 shown in FIG. 10 corresponds to the geometry shown in FIG. 9 in accordance with an exemplary embodiment. The triangle 515 includes a first side 518, a second side 520 and a third side 525. The first side 518 is the length from a fixed position on the adjustable needle guide 170 to the target. The first side 518 corresponds to expected guide path 508 shown in FIG. 9. According to an exemplary embodiment, the first side 518 may extend from the pivot 180 to a center of the target 521. The center of the target 521 may, for instance be the position where the visual indicator 506 is positioned. The second side 520 corresponds to the base guide line 510 shown in FIG. 9. The first side 518 and the second side 520 of the triangle 515 are in the same relative positions with respect to each other as the expected guide line 508 and the base guide line 510 in FIG. 9. The second side 520 may extend from a fixed position on the flexible needle guide to a position where a line perpendicular to the second side 520 intersects with the center of the target 521. The third line 525 is perpendicular to the second side 520 and extends from the second side 520 to the center of the target 521. The processor 116 may be configured to calculate a first length of the first side 518 and a second length of the second side 520 based on lateral information and depth information from the ultrasound image 502 and the known relationship of the flexible needle guide 170 to the image 502. The processor 116 may determine the length of the third line 525 based on the lateral information and the depth information in the image 502. Based on the triangle 513 shown in FIG. 10, the processor 116 may be configured to calculate the angle 513 using a trigonometric relationship based on any two of the sides of the triangle 513. After identifying the angle 513 in the triangle 515 (which is the same as the angle 513 shown on FIG. 9), the processor 116 may calculate a setting for the adjustable needle guide 170. For example, the angle 513 between the base guide line 510 and the expected guide line 508 may represent the angle that the adjustable needle guide needs to be adjusted from the position represented by the base guide line 510. For example, if the angle 513 is 10 degrees, then the adjustable needle guide 170 needs to be adjusted 10 degrees from the position corresponding to the base guide line 510. Those skilled in the art should appreciate that 10 degrees is merely an exemplary angle and the angular difference between the base guide line 510 and the expected guide line 508 may be different amounts according to various embodiments.

The processor 116 may be configured to calculate the setting for the adjustable needle guide 170 using other geometrical technique using the known position of the adjustable needle guide 170 with respect to the image 502. For example, according to an embodiment, the processor 116 may be configured to calculate a setting of the adjustable needle guide 170 by identifying the position of the target 504 in the image 502 and then determining an angle of the expected guide line 508 based on the known relative positions of the image 502 and the adjustable needle guide 170. The processor 116 may, for instance, calculate the relative angle of the expected guide line 508 with respect to the adjustable needle guide 170 and use that relative angle to determine the setting.

The processor 116 may be configured to convert an angle, such as the angle 513, into a setting for the adjustable needle guide 170. For example, the processor 116 may access geometrical information about the adjustable needle guide 170 from memory or a look-up table and then use the geometrical information to calculate the setting. For example, according to an exemplary embodiment described with respect to FIGS. 9 and 10, after calculating the angle 513, the processor 116 may be configured to determine the setting for the adjustable needle guide 170. According to an embodiment where the angle 513 is 10 degrees, the processor 116 determine the setting by adjusting the base setting by 10 degrees in order to determine the setting for the adjustable needle guide 170. The setting may be an angle measurement for the adjustable needle guide, or the setting may be an indication of one of a plurality of angular positions for the adjustable needle guide. It should be appreciated that 10 degrees is an exemplary angle and that the angle may be any value according to various embodiments.

Next, at step 310 the processor 116 is configured to display the setting on the display device 118. For example, the processor 116 may be configured to display an angle measurement (such as 5 degrees, 10 degrees, 15 degrees, etc.) for the adjustable needle guide or the processor 116 may be configured go display one of a plurality of angular positions for the adjustable needle guide. As discussed previously, the adjustable needle guide 170 may include an adjustable portion such as the angle guide 182. When using an adjustable needle guide with an angle guide such as the adjustable needle guide 170, the processor 116 may be configured to display the setting as an angle measurement. For example, FIG. 4 includes an angle measurement 450 displayed on the display device 118 in accordance with an embodiment. The angle measurement 450 says, “Needle Guide: 45°” in accordance with an embodiment. As such, the clinician would know to set the adjustable needle guide 170 to a position where the angle measurement indicates “45°.” It should be appreciated by those skilled in the art that 45° is just one exemplary example of an angle measurement and that different angle measurements may be presented according to various embodiments and clinical situations.

FIG. 5 includes an example where the setting is one of a plurality of settings. For example, FIG. 5 displays the setting 550, which states, “Needle Guide: Position C.” The screenshot 500 corresponds to an adjustable needle guide such as the adjustable needle guide 270 shown in FIG. 8, which includes a plurality of different settings (e.g., A, B, C, and D in the adjustable needle guide 270). The setting 550 indicates to the clinician that the adjustable needle guide 270 should be set to position C. As described previously, in other embodiments, the processor may be configured to display a number or other indicia to designate one of a plurality of settings when displaying the setting at step 310.

By providing a setting for the adjustable needle guide (170, 270) based on a user-selected target, the invention enable a clinician to quickly and accurately set-up the adjustable needle guide (170, 270) in order to accurately guide a needle to the target. Providing a setting for the adjustable needle guide (170, 270) based on a target in the image reduces the total time required to perform an ultrasound-guided procedure involving a needle, such as obtaining a biopsy or administering a nerve block. Additionally, by providing a setting for the adjustable needle guide (170, 270) based on a target present in the image obtained during the procedure, the invention reduces the number of attempts it will take a clinician to accurately position the needle in the target anatomy within the patient. The present invention also permits the user to keep the ultrasound probe 106 in good acoustic contact with the patient while inserting the needle since the setting for the adjustable needle guide (170, 270) was determined based on a target identified from the image. Having good acoustic contact helps to ensure high-quality imaging while inserting the needle through the adjustable needle guide (170, 270).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method for determining a setting of an adjustable needle guide that is configured to be used with an ultrasound imaging system comprising an ultrasound probe, processor, and a display device, wherein the adjustable needle guide is configured to be attached to the ultrasound probe, the method comprising:
acquiring an image including a target with the ultrasound probe;
displaying the image on the display device;
receiving an input, via the user interface, identifying the target in the image;
automatically calculating, with the processor, the setting for the adjustable needle guide based on the identified target in the image, wherein the setting is at least one of an angle measurement for the adjustable needle guide or one of a plurality of angular positions for the adjustable needle guide, and wherein the setting is configured to position the adjustable needle guide to guide a needle to the target; and
displaying the setting for the adjustable needle guide on the display device.

2. The method of claim 1, wherein the user interface comprises a touch panel that is a component of a touchscreen, and wherein the input comprises a touch input.

3. The method of claim 1, wherein said receiving the input comprises receiving an input via one of a mouse, a trackball, or a trackpad positioning a visual indicator on the target in the image.

4. The method of claim 1, further comprising displaying an expected guide line on the image after receiving the input identifying the target in the image, wherein the expected guide line represents an expected path of a needle with the adjustable needle guide adjusted to the setting.

5. The method of claim 4, wherein the user interface comprises a touch panel that is a component of a touchscreen, and wherein the expected guide line is displayed during the process of said receiving the touch input via the touch panel.

6. The method of claim 4, further comprising showing a base guide line on the image, wherein the base guide line represents a calibrated position with respect to the adjustable needle guide.

7. The method of claim 1, wherein said automatically calculating the setting comprises calculating an angle between the base guide line and the expected guide line with the processor.

8. The method of claim 1, wherein the plurality of angular positions is a plurality of indexed angular positions.

9. An ultrasound imaging system comprising:
an ultrasound probe;
a display device;
a user interface; and
a processor, wherein the processor is configured to:
control the ultrasound probe to acquire an image including a target;
display the image on the display device;
receive an input, via the user interface, identifying the target in the image;
automatically calculate a setting for an adjustable needle guide that is configured to be attached to the ultrasound probe based on the identified target in the image, wherein the setting is at least one of an angle measurement for the adjustable needle guide or an indication of one of a plurality of angular positions for the adjustable needle guide; and
display the setting on the display device.

10. The ultrasound imaging system of claim 9, wherein the processor is configured to display the angle measurement on the display device.

11. The ultrasound imaging system of claim 9, wherein the processor is configured to display the indication of one of the plurality of angular positions on the display device.

12. The ultrasound imaging system of claim 9, wherein the user interface comprises one of a mouse, a trackball, or a trackpad, and wherein the user input comprises using the user interface to position a cursor on the target in the image.

13. The ultrasound imaging system of claim 9, wherein the user interface comprises a touch panel that is a first component of a touchscreen, and wherein the display device comprises a display screen that is second component of the touchscreen.

14. The ultrasound imaging system of claim 9, wherein the processor is configured to display a base guide line on the image, wherein the base guide line is a calibrated position with respect to the adjustable needle guide.

15. The ultrasound imaging system of claim 14, wherein the processor is configured to display an expected guide line on the image after receiving the input identifying the target in the image, wherein the expected guide line represents an expected path of the needle after the adjustable needle guide has been adjusted to the setting.

16. The ultrasound imaging system of claim 9, wherein the processor is configured to display an expected guide line on the image after receiving the input identifying the target in the image, wherein the expected guide line represents an expected path of a needle after the adjustable needle guide has been adjusted to the setting.

17. The ultrasound imaging system of claim 9, wherein the setting is one of the plurality of angular positions.
